(19) United States
(12) Patent Application Publication
(10) Pub. No.: US 2021/0041853 A1
(43) Pub. Date: Feb. 11, 2021

(54) Systems, Devices, and Methods for Generating a Model of a Vascular Network, and for Analyzing and/or Treatment Planning Related to Thereof

(71) Applicants: Emory University, Atlanta, GA (US); Children's Healthcare of Atlanta, Inc., Atlanta, GA (US)

(72) Inventors: Vahid Serpooshan, Atlanta, GA (US); Holly Bauser-Heaton, Atlanta, GA (US); Martin L. Tomov, Decatur, GA (US)

(21) Appl. No.: 16/987,321

(22) Filed: Aug. 6, 2020

Related U.S. Application Data
(60) Provisional application No. 62/883,305, filed on Aug. 6, 2019.

(51) Int. Cl. G05B 19/4099; A61B 34/10; A61B 5/02; B33Y 10/00; B33Y 50/02; B33Y 80/00; G16H 50/50; G16H 30/40

(52) U.S. Cl. CPC G05B 19/4099; A61B 34/10; A61B 5/02007; B33Y 10/00; G05B 2219/49023; B33Y 80/00; G16H 50/50; G16H 30/40; G05B 2219/35151; B33Y 50/02

(57) ABSTRACT

The systems and methods are provided that can efficiently and accurately generate 3D printed vascular models of a vascular network, including stenotic pulmonary arteries, capable of vascular perfusion. The method may include acquiring image(s) of an anatomy of interest that includes a target area. The method may further include generating a geometric model of a phantom of a vascular network to be bioprinted using the image(s). The phantom may include vascular segment(s), inlet(s), and outlet(s). Each inlet and each outlet may communicate with at least one vascular segment. The method may include generating a geometric model of a bioreactor to be 3D printed based on the geometric model of the phantom using one or more of assembly parameters, phantom parameters, or any combination thereof. The bioreactor model may include inlet(s), outlet(s), a chamber in which the phantom is disposed, an outer housing, and an interface bordering the chamber.

### CROSS-REFERENCE TO RELATED APPLICATIONS

This application claims the benefit of U.S. Provisional Application No. 62/883,305 filed Aug. 6, 2019. The entirety of this application is hereby incorporated by reference for all purposes.

### STATEMENT REGARDING FEDERALLY SPONSORED RESEARCH OR DEVELOPMENT

This invention was made with government support under HL127295 awarded by the National Institutes of Health. The government has certain rights in the invention.

### BACKGROUND

Recently, additive manufacturing, and in particular, 3D printing techniques have emerged as robust engineering tools to create a variety of high-fidelity 3D models. For example, conventional 3D printing methods can generally generate synthetic (non-biological) constructs and recent 3D bioprinting methods can fabricate live, functional tissue and organ mimics for a variety of research and training applications in the basic sciences and medical fields.

When leveraged towards biomedical and clinical applications, 3D printing and bioprinting can generate accurate models of patient pathologies based on commonly used medical imaging tools such as magnetic resonance imaging (MM), computed tomography (CT), or X-ray angiography (XA). 3D printed models can aid in the visualization of the precise 3D configuration of collaterals and native pulmonary arteries. They can therefore improve the understanding of the pathophysiology of diseases related thereto, such as cardiovascular conditions, including pulmonary artery stenosis (e.g., pulmonary atresia (PA) with ventricular septal defect (VS) and major aortopulmonary collateral arteries (MAPCAs), and aid in the development and optimization of surgical treatments.

However, conventional 2D and 3D vascular models generally do not accurately represent the dynamic and complex tissue microenvironment. Also, many currently available 3D in vitro models, such as 3D in vitro models of stenotic pulmonary arteries, have not been capable of vascular perfusion_and/or have been unable to sustain in vivo-like rates.

### SUMMARY

Thus, there is need for accurate 3D vascular models that are capable of vascular perfusion, and that are also capable of supporting cell cultures long-term, and that allow functional assays_to be performed.

The disclosure relates to systems and methods that can efficiently and accurately generate 3D vascular models of a vascular network, including stenotic pulmonary arteries, capable of vascular perfusion. The systems and methods relate to generated model of and/or produced perfusable assembly that includes a phantom of a vascular network disposed within a bioreactor.

In some embodiments, the methods may include a method for generating a 3D perfusable assembly of a vascular network. The method may include acquiring one or more images of an anatomy of interest, the anatomy of interest including a target area. The method may further include generating a geometric model of a phantom of a vascular network using the one or more images. The phantom may include one or more vascular segments, one or more inlets, and one or more outlets. Each inlet and each outlet may communicate with at least one vascular segment. In some embodiments, the method may further include generating a geometric model of a bioreactor based on the geometric model of the phantom using one or more of assembly parameters, phantom parameters, or any combination thereof. The bioreactor model may include one or more inlets, one or more outlets, a chamber in which the phantom is disposed, an outer housing, and an interface bordering the chamber.

In some embodiments, the systems may include a system for generating a 3D perfusable assembly of a vascular network. In some embodiments, the system may include one or more processors; and one or more hardware storage devices having stored thereon computer-executable instructions. The instructions may be executable by the one or more processors to cause the computing system to perform at least acquiring one or more images of an anatomy of interest, the anatomy of interest including a target area. The one or more processors may be further configured to cause the computing system to perform at least generating a geometric model of a phantom of a vascular network using the one or more images. The phantom may include one or more vascular segments, one or more inlets, and one or more outlets. Each inlet and each outlet may communicate with at least one vascular segment. In some embodiments, the one or more processors may also be further configured to cause the computing system to perform at least generating a geometric model of a bioreactor based on the geometric model of the phantom using one or more of assembly parameters, phantom parameters, or any combination thereof. The bioreactor model may include one or more inlets, one or more outlets, a chamber in which the phantom is disposed, an outer housing, and an interface bordering the chamber.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows an example of a system for generating a perfusable vascular assembly according to embodiments;

FIG. 2 shows a method of producing a perfusable vascular assembly using image data according to embodiments;

FIG. 3 shows an example of an image showing a target area according to embodiments;

FIG. 4 shows a method of generating a geometrical model of a phantom according to embodiments;

FIG. 5A shows an example of a geometrical model of the target area shown in FIG. 3B according to embodiments;

FIG. 5B shows an example of a generating a geometric model of the vascular network provided in the target area shown in FIG. 5A according to embodiments;

FIG. 5C shows an example of a geometric model of the vascular network determined from FIG. 5B according to embodiments;

FIG. 5D shows an example of a produced phantom using the geometric model shown in FIG. 5C using a three dimensional printer according to embodiments;

FIG. 5E shows an example of a bioprinted phantom using the geometric model shown in FIG. 5C according to embodiments;

FIG. 6 shows the resulting generated geometric model of the phantom for the vascular network shown in FIG. 5C according to embodiments;

FIG. 7 shows a method of generating a geometrical model of a bioreactor according to embodiments;

FIG. 8A shows an example of a geometric model of a bioreactor for the phantom shown in FIG. 6 according to embodiments;

FIG. 8B shows the resulting generated geometric model of the bioreactor shown in FIG. 8A;

FIG. 9A shows the resulting geometric model of the assembly of the phantom shown in FIG. 6 and the bioreactor shown in FIG. 8B;

FIG. 9B shows a partial, exploded view of the assembly shown in FIG. 9A;

FIG. 10A shows an example of a produced assembly of the model shown in FIG. 9A according to embodiments;

FIG. 10B shows an example of a perfusion system and the produced assembly shown in FIG. 10A according to embodiments;

FIG. 10C shows an example of another perfusion system for a plurality of produced assemblies shown in FIG. 10A according to embodiments;

FIG. 11A shows an example of using the produced assembly to test an anastomotic procedure;

FIG. 11B shows an example of the perfusable phantom generated to model the target area shown in FIG. 3; and

FIG. 12 shows a block diagram illustrating an example of a computing system according to embodiments.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The methods, systems, and devices of the disclosure may be related to processing at least medical images (e.g., of a patient) to generate pathology-specific perfusable assemblies for use in clinical and research applications, such as development and optimization of surgical treatments for a patient and/or pathology.

In some embodiments, the perfusable assembly may include a bioprinted phantom disposed within a three-dimensional printed bioreactor. The phantom may be a three-dimensional functional phantom of a vascular network. For example, the phantom may include a (simplified) geometric model of a portion of a vascular network determined from the medical images and clinical data disposed within a phantom housing.

In some embodiments, the phantom may include one or more perfusable vascular segments (e.g., lumens) having inlet(s)/outlet(s) connected to respective one or more inlets and/or outlets of the bioreactor. In some embodiments, the one or more vascular segments may be a branched network. By way of example, the phantom may represent healthy or diseased regions of arteries, veins, valves, ducts, interstitial tissue, among others, or any combination thereof. For example, the disease regions may include but is not limited to pulmonary artery stenosis (e.g., PA with MAPCAs), pulmonary vein stenosis (PVS), other cardiovascular conditions of a vein and/or artery, conditions related to ducts, among others, or any combination thereof.

In some embodiments, the phantom may further include a segment (conduit) at a location of a proposed treatment (e.g., recanalization procedure) within the vascular network. The conduit may include but is not limited to a location where a (proposed) stent may be implant, graft may be implanted, other clinical interventions may be performed, or a combination thereof.

In some embodiments, the bioreactor may include a chamber in which the phantom may be disposed and at least one inlet and at least one outlet. The bioreactor may be selected from a plurality of bioreactor templates using the generated model of the phantom (e.g., dimensions, size, vascular network geometry, among others), user settings (e.g., selected perfusion rates), among others, or any combination thereof.

In some embodiments, the perfusable assembly may be produced using one or more three dimensional printers. For example, the three-dimensional printers may be any automated, computer-aided three-dimensional prototyping printers, including a three-dimensional printer having bioprinting capabilities. For example, the phantom may be produced by a bio-printer using bioink. The bio-ink may be any liquid, semi-solid, or solid composition comprising a plurality of cells. In some embodiments. the bioink may include cell solutions, cell aggregates, cell-comprising gels, multicellular bodies, tissues, among others, or a combination thereof. In some embodiments, the bioink may also include a support material, such as non-cellular materials that can provide specific biomechanical properties that enable bioprinting.

In some embodiments, the produced perfusable vascular assembly may be designed to allow imaging guidance, maintain hydrogel stiffness comparable to in vivo conditions, allow for homeostatic flow preanastomosis and postanastomosis, among others, or a combination thereof. The produced perfusable vascular assembly may be used for one or more analyses. For example, the one or more analyses may include disease modeling of the (e.g., cardiovascular) conditions in vitro, drug screening, surgical intervention development and/or planning, other medical and/or surgical interventions, among others, or a combination thereof. By way of example, for PA and/or for PVS, the produced assembly may be used to test a proposed vascular anastomosis (unifocalization) procedure to recanalize the vascular network representing an atretic artery in PA, to simulate a stent-based expansion of stenotic vein in the PVS assembly, among others, or a combination thereof.

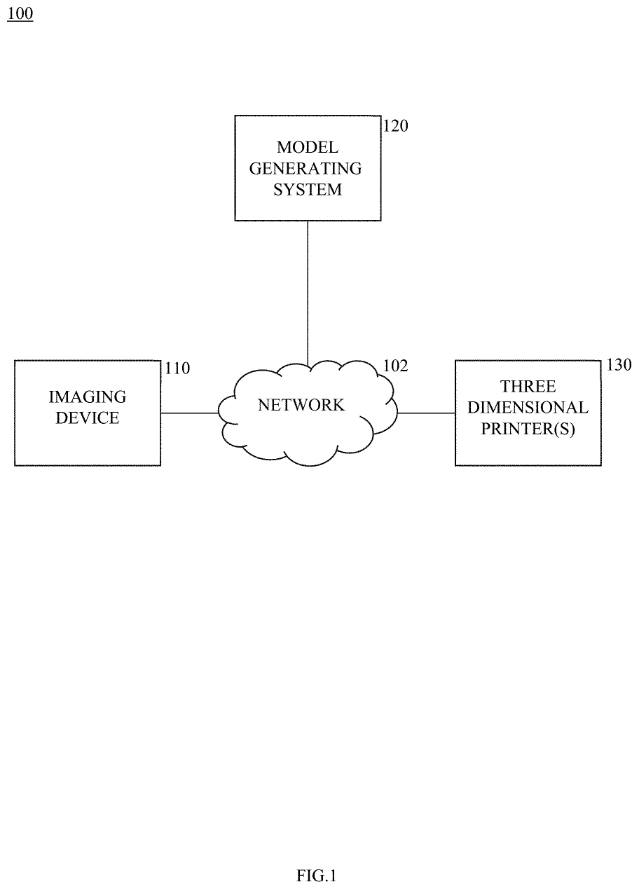

FIG. 1 illustrates a system 100 for generating a perfusable assembly, according to some embodiments. In some embodiments, an imaging device 110 may be used to acquire one or more images of an anatomy of interest. The imaging device 110 may include but is not limited to magnetic Resonance Imaging (MR) scanner, a Computed Tomography (CT) scanner, a Positron Emission Tomography (PET) scanner, X-Ray Angiography (XA), an ultrasound device, among others, or any combination thereof. For example, in some embodiments, the images may be acquired using multiple modalities and aggregated to provide various types of image data corresponding to the anatomy/region of interest.

In some embodiments, an assembly/model generating device 120, such as a workstation, personal computer, central processing system, among others, or any combination thereof, may receive the image data from the imaging device 110 directly or indirectly via a computer network 102. This computer network 102 may be configured using a variety of hardware platforms. For example, the computer network 102 may be implemented using the IEEE 802.3 (Ethernet) or IEEE 802.12 (wireless) networking technologies, either separately or in combination. The computer network 102 may be implemented with a variety of communication tools including, for example, TCP/IP suite of protocols. In some embodiments, the computer network 102 may be the Internet. A virtual private network (VPN) may be used to extend a private network across the computer network 102. In some embodiments, the image data may be stored on a database (e.g., DICOM, PACs, EMR, etc.) on the network 102 from which the generating device 120 may receive the image data.

The generating device 120 may generate the three-dimensional geometric model data corresponding to the perfusable assembly and one or more specifications (bioink compositions, print parameters, etc.) related thereto using the image data, one or more assembly parameters, one or more stored bioreactor templates, among others, or a combination thereof.

In some embodiments, the one or more assembly parameters may include one or more phantom parameters, one or more bioreactor parameters, one or more analysis parameters, among others, or a combination thereof. For example, the one or more phantom parameters may include but is not limited to bioink compositions for the phantom, diameters of segments of phantom, layer height, other mechanical properties, among others, or a combination thereof. In some embodiments, the one or more analysis parameters may include but is not limited to type of analysis to be performed (e.g., cell types to be analyses, treatment to be analyzed, etc.), one or more perfusion parameters (e.g., desired perfusion rate), among others, or a combination thereof. In some embodiments, the generating device 120 may store one or more bioreactor templates to be used as a reference for generating the model of the phantom, the model of the bioreactor, among others, or a combination thereof.

The generating device 120 may be operably coupled to a user device which allows clinicians to provide inputs, such as one or more assembly parameters. to the assembly generating device 120. In some embodiments, one or more of the assembly parameters may be stored, default settings. For example, the perfusion parameters, the bioink composition, the diameter of the inlet(s)/outlet(s), among others, or a combination thereof may be stored. In some embodiments, the one or more settings may be associated with the analysis to be performed, bioreactor design, among others, or a combination thereof.

For example, the bioink may include but is not limited gelatin methacryoyl (gelMA) (e.g., 10-20% (w/v) gelMA), Pluronic (e.g., 27% (w/v)), among others, or a combination thereof.

Once generated, the three-dimensional geometric model data (e.g., CAD files) for the assembly, the phantom, and/or the bioreactor and associated specifications (e.g., bioink composition) may be sent to the one or more three-dimensional printers 130 to produce the phantom, the bioreactor and/or the assembly. The one or more specifications transmitted to the printer(s) along with the geometrical model(s) may include dimensions, print parameters (e.g., bioink compositions, extrusion pressure, UV crosslinking time, layer height, nozzle diameter, print speed, etc.), among others, or any combination thereof.

For example, the one or more three-dimensional printers may include or have capabilities of a bioprinter, a resin 3D printer, among others, or a combination thereof. For example, a bioprinter may be configured to produce the phantom. The bioprinter may include but is not limited to a 2 nozzle extrusion bioprinter, such as BioAssemblyBot™ bioprinter, BioX™ bioprinter and the Allevi 3 bioprinter. The resin 3D printer may be configured to produce the bioreactor. For example, the resin 3D printer may include but is not limited to a 3D printer capable of light-based cross-linking of resins via a digital light processing (DLP) or stereolithography (SLA), such as Form 3 Low Force Stereolithography (LFS)™ 3D printer.

After which the assembly is produced, one or more of the assemblies may be attached to a perfusion system for testing and/or analysis. For example, the perfusion system may include peripheral accessories and/or tools (e.g., pumps, microscopes, etc.) to test the phantom disposed within the assembly with respect to one or more treatments, one or more analyses, among others, or a combination thereof. For example, the perfusion system may include an external media source and perfusion pumps (e.g., peristaltic, constant flow, syringe, etc.) so as to mimic a vascular system when the assembly is connected. The treatments may include but are not limited to surgical tools and/or implantation devices such as catheters, wire, stents, valves, flow sensors, among others, or a combination thereof. The analyses may include but is not limited to metabolic, metabolomic, bioprofiling, soluble molecule analysis assays, among others, or any combination thereof. For example, a microscope may be used to image live and/or fixed cells within the produced phantom, or supernatant collected to perform metabolomics and/or cell bioprofiling of the live cultures.

Although the systems/devices of the system 100 are shown as being directly connected, the systems/devices may be indirectly connected to one or more of the other systems/devices of the system 100. In some embodiments, a system/device may be only directly connected to one or more of the other systems/devices of the system 100.

It is also to be understood that the system 100 may omit any of the devices illustrated and/or may include additional systems and/or devices not shown. It is also to be understood that more than one device and/or system may be part of the system 100 although one of each device and/or system is illustrated in the system 100. It is further to be understood that each of the plurality of devices and/or systems may be different or may be the same. For example, one or more of the devices of the devices may be hosted at any of the other devices.

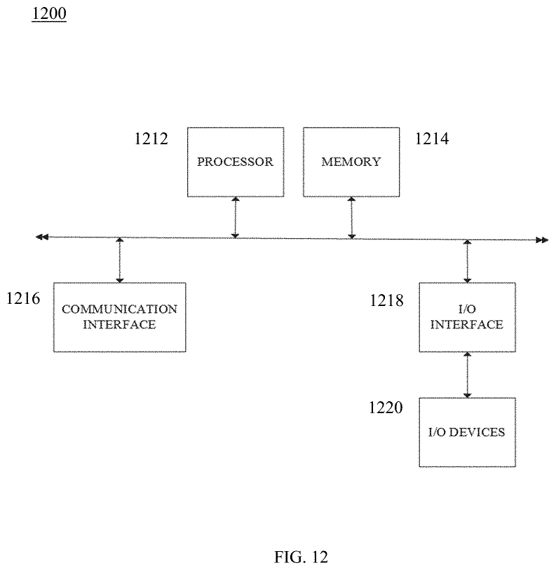

In some embodiments, any of the devices of the system 100, for example, the devices 120 and 130, may include a non-transitory computer-readable medium storing program instructions thereon that is operable on a user device. A user device may be any type of mobile terminal, fixed terminal, or portable terminal including a mobile handset, station, unit, device, multimedia computer, multimedia tablet, Internet node, communicator, desktop computer, laptop computer, notebook computer, netbook computer, tablet computer, personal communication system (PCS) device, wearable computer (e.g., smart watch), or any combination thereof, including the accessories and peripherals of these devices, or any combination thereof. FIG. 12 shows an example of a user device.

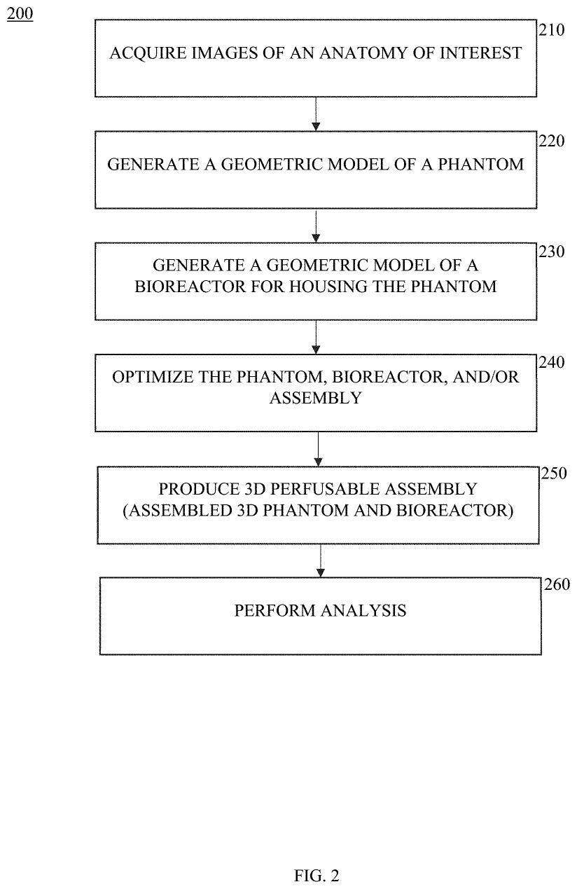

FIG. 2 shows a method 200 of producing a perfusion assembly including the phantom and bioreactor according to embodiments. Unless stated otherwise as apparent from the following discussion, it will be appreciated that terms such as “encoding,” “generating,” “determining,” “displaying,” “obtaining,” “applying,” “processing,” “computing,” “selecting,” “receiving,” “detecting,” “classifying,” “calculating,” “quantifying,” “outputting,” “acquiring,” “analyzing,” “retrieving,” “inputting,” “assessing,” “performing,” “producing,” “optimizing,” “updating,” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. The system for carrying out the embodiments of the methods disclosed herein is not limited to the systems shown in FIGS. 1 and 12. Other systems may also be used.

The methods of the disclosure are not limited to the steps described herein. The steps may be individually modified or omitted, as well as additional steps may be added. It will be also understood that at least some of the steps may be performed in parallel.

The method 200 may include a step 210 of acquiring and/or receiving image(s) of an anatomy of interest, for example, from the imaging device 110. The anatomy of interest may include a region of interest. For example, the region of interest may be identified by the user. The region of interest may include but is not limited to stenotic area, pulmonary atresia, among others.

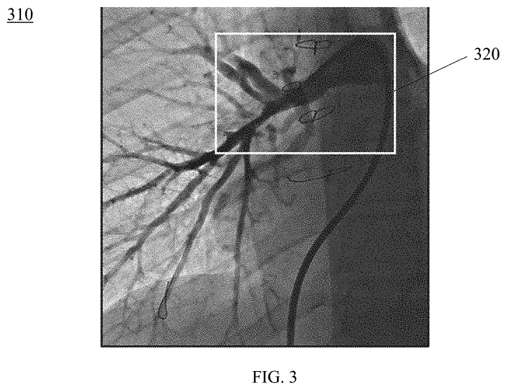

FIG. 3 shows an example 310 of image of an anatomy of interest having a region of interest 320. The region of interest 320 can be used to determine the geometric model of the phantom of the vascular network. In this example, the image is of a portion of a heart of a subject who has PAS.

In the step 210, one or more assembly parameters, patient/clinical data, among others, or a combination thereof, may be also be received. The assembly parameter(s) may include one or more analysis parameter(s), phantom parameter(s), bioreactor parameter(s), among others, or any combination thereof. The phantom parameters may include but is not limited to the type of vascular network being modeled (e.g., stenotic area, PA, etc.), number of inlet(s)/outlet(s), diameter of segment(s), bioink compositions, among others, or a combination thereof.

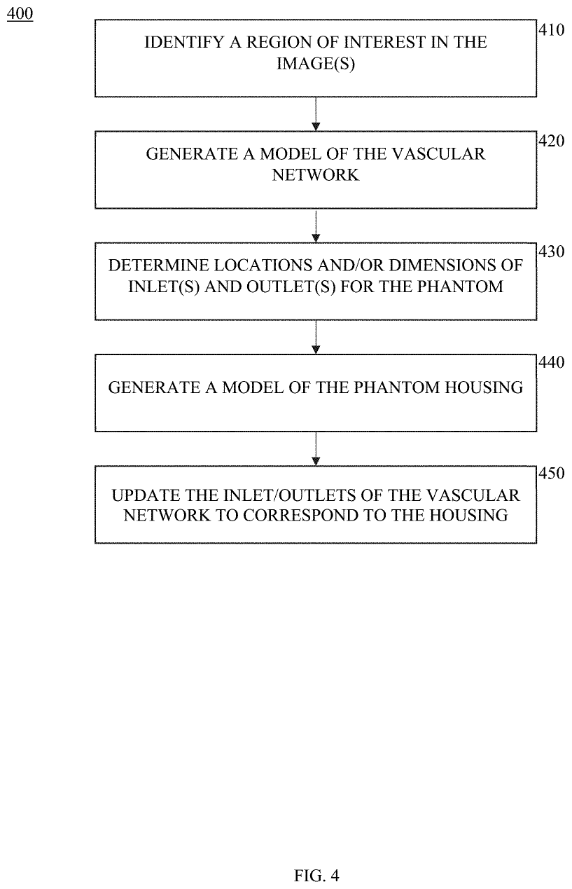

Next, the method 200 may include a step 220 of generating a geometric model of the phantom using the image data. In some embodiments, the phantom may include a geometric model of the vascular network, one or more inlets, one or more outlets, and housing. The geometric model of the vascular network may be a simplified model of a portion of a target area within the anatomy of interest. In some embodiments, the geometric model may be generated using a method 400 shown in FIG. 4. In some embodiments, the geometric model may be generated using other methods.

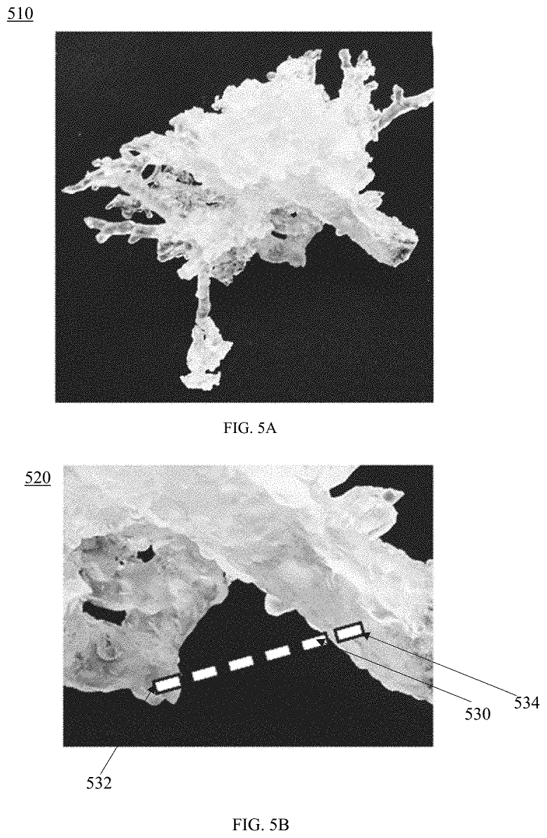

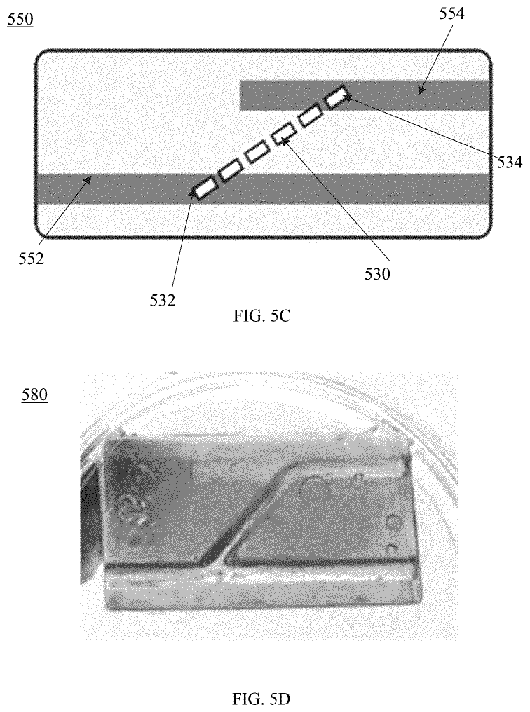

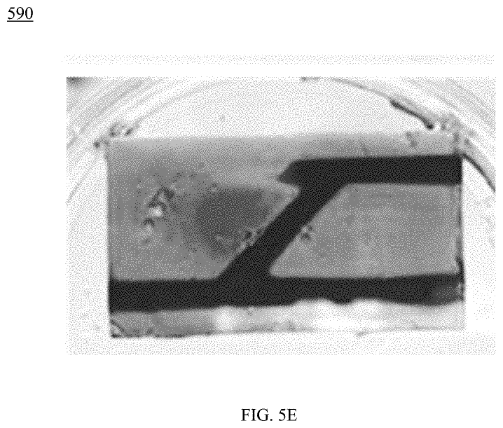

FIG. 4 shows an example of the 400 of generating a geometric model of the phantom of the vascular network according to some embodiments. In some embodiments, the method 400 may include a step 410 of identifying a region that includes the target area in the image(s) of an anatomy of interest. The step 410 may be performed manually or automatically. By way of example, FIG. 5A shows an example 510 of a model of the vascular network corresponding to the region 320 that includes the target area shown in FIG. 3.

Next, the method 400 may include step 420 of generating a geometric model of at least the vascular network according to some embodiments. In some embodiments, the step 420 may include removing extraneous vascular segments (e.g., cardiovascular vessels) that are not directly related to the target area (e.g., stenotic vein/artery) so that only the segments directly connected with respect to the target area remain. For example, extraneous vascular segments may include those segments that are not in direct fluid communication (e.g., flow region) within the target area and/or inlet(s)/outlet(s). For example, the step 420 may include segmenting a region including the target area with respect to a zero plane, disposed in a middle of the region or a side of the region to determine one or more segments that is in an active flow region of the target area, is one or more segments that are to be connected in the target area by a segment (e.g., conduit) representing a treatment, has a size and/or vessel diameter within a set range, among others, or a combination thereof.

By way of example, FIG. 5B shows an example 520 of the target area of the model 510 shown in FIG. 5A with a proposed conduit. In this example, the target area may be segmented starting at the side closest to an inlet 532 of the segment 530 to the side of the outlet 534 of the segment 530 to determine the active flow region with respect to the conduit 530. The target area may be cropped removing any segments not in the active flow region or not in treatment region (e.g., potential targets for anastomosis) may be removed.

After the vascular segments in the active flow region and/or the segment for proposed treatment have been determined, the geometric model specific to those segments within the target area may be simplified so that the remaining segments are in a single plane. For example, the vascular network representing the active flow region and/or the segment(s) representing a proposed treatment site within the target area may be modified to be in a single plane. FIG. 5C shows an example 550 of the generated geometric model of the (simplified) vascular network shown in FIGS. 5A and 5B. As shown, the segments 552 and 554 are in the active flow region of the target area/proposed conduit 530. FIGS. 5D and 5E show examples 580 and 590 of a phantom generated based on the geometrical model shown in FIG. 5C, without and with bioink, respectively. In these examples, the phantom includes an occluded (atretic MAPCA) segment and an open (PA) vessel segment.

After, the method 400 may include a step 430 of determining locations and/or dimensions of the one or more inlet(s) and/or outlet(s) of the vascular network for the phantom in some embodiments. For example, the diameters of the inlet(s) and/or the outlet(s) may be based on the native vasculature. By way of example, the diameters may be determined from the patient data where vessel diameter can be measured. For example, the diameters of the inlet(s) and/or outlet(s) may correspond to the diameter of the segment that has been identified to be of interest.

Next, the method 400 may include a step 440 of generating a geometric model of the phantom housing in some embodiments. For example, the phantom housing may be a rectangular shaped block that encases the vascular network when the phantom is bioprinted. The step 440 may include the dimensions of the housing (e.g., thickness, length, and width). For example, the length and width of the housing may be sufficient to encompass the geometric model of the vascular network. The thickness may depend on the desired flow rate (i.e., perfusion parameters). For example, the thickness may correspond to the minimal thickness that could permit the desired flow rate.

In some embodiments, the method 400 may include a step 450 of modifying the vascular network to correspond to the phantom housing. For example, the inlet(s) and outlet(s) of the vascular network determined in step 430 may be modified to correspond to the dimensions of the phantom housing, bioreactor parameter(s), one or more bioreactor templates (e.g., standard inlet/outlet diameter), among others, or a combination thereof. For example, the segments having inlet(s) and outlet(s) may be extended to the edge of the housing so that the corresponding openings disposed at respective ends corresponding to the inlet(s) and outlet(s) are disposed at the edge. In some embodiments, the diameter of the openings may be updated, if needed, to correspond to the diameters included in the bioreactor parameters.

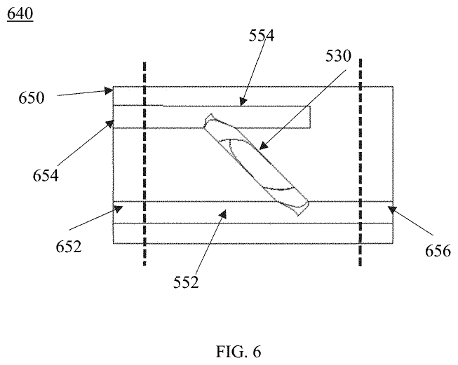

FIG. 6 shows an example 640 of the vascular network 550 shown in FIG. 5C (from a different side) encased in the phantom housing 650. In this example, the model of the vascular network shown between the dashed lines corresponds to the vascular network determined in step 440. As shown in this example, the length of the segment 554 may be extended in one direction towards a side so that the opening 654 corresponding to the outlet is disposed at the edge of the housing on a first side; and the length of the segment 552 may be extended in both directions towards the opposing sides so that the opening 652 corresponding to the outlet is disposed at the edge of the housing on the first side and the opening 656 corresponding to the inlet is disposed at the edge of the housing on a second side. In this example, the inlet is disposed on the opposing side. In other examples, the inlet may be disposed on any side other than the first side (e.g., side in which the corresponding outlet is disposed), such as a side perpendicular to the first side.

After the step 450, the geometric model of the phantom of the vascular network may be generated. The geometric model may be used by a bioprinter to produce the phantom.

After the geometric model of phantom is generated, the method 200 may include a step 230 of generating a geometric model of a bioreactor for storing the phantom during the testing/analysis. In some embodiments, the bioreactor design may be determined using one or more of the stored templates based on the parameters of the generated phantom/vascular network model (step 220), the one or more testing parameters, the bioreactor parameters, patient/clinical data, among others, or a combination thereof.

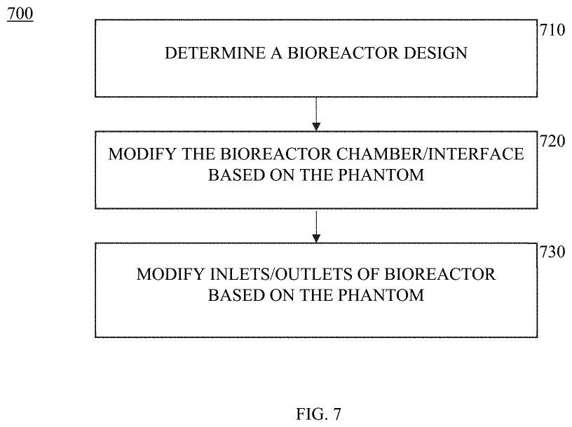

In some embodiments, the geometric model of the bioreactor may be generated using a method 700 shown in FIG. 7. In some embodiments, the geometric model of the bioreactor may be generated using other methods and/or a prefabricated bioreactor may be used.

In some embodiments, the method 700 may include a step 710 of determining a bioreactor design. In some embodiments, the bioreactor may a chamber to hold the phantom and to be in fluid communication with the phantom, an interface that borders the chamber, an outer housing, a support frame disposed between the interface and the outer housing, inlet(s) and outlet(s) in fluid communication with the chamber, barbed connector(s)/adapter(s) disposed at the respective openings of inlet(s)/outlet(s), and a modular cover having a window corresponding to the chamber. In some embodiments, the bioreactor may further include a model of at least portion of or related to the anatomy of interest that is not a part of the vascular network of the phantom, for example, disposed within the chamber and in communication with the inlet(s) and/or outlet(s). For example, the model may include another portion of the anatomy of interest adjacent to or connected to the vascular network.

In some embodiments, the model of the portion of the anatomy of interest may be used to test phantom function when assembled. By way of example, the portion of anatomy of interest may include but is not limited to a heart chamber (or portion thereof) that can be used when the phantom is assembled with the bioreactor to simulate pressure drop; a vessel extension configured to build up pressure when the phantom is assembled with the bioreactor to simulate pressure drop; among others, or a combination thereof. In some embodiments, the portion of the anatomy of interest may be based on patient specific image data or may be standardized based on image data from a plurality of patients, among others, or a combination the.

In some embodiments, the bioreactor design may be selected from a plurality of stored bioreactor design templates, for example, using properties of the generated model of the phantom (e.g., the size of the phantom, number of the inlet(s)/outlet(s), position of the inlet(s)/outlet(s), etc.), flow rates based on in vivo data, patient data (e.g., vessel diameter), cell type to be seeded into the produced phantom, mechanical properties of the bioink, analyses to be performed, among others, or a combination thereof. The bioreactor design templates may differ in size (e.g., dimensions of chamber), inlet/outlet dimensions (e.g., diameter), number of inlet(s)/outlet(s), inlet/outlet configuration (e.g., position within bioreactor), cover, a portion of the anatomy of interest, analyses to be performed, barbed connections/adapters, among others, or any combination thereof.

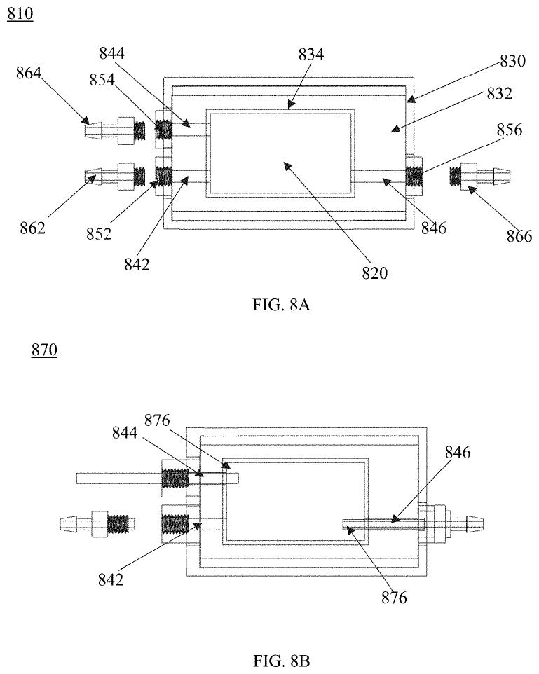

By way of example, FIG. 8A shows an example of a bioreactor 810 determined for the phantom determined in FIG. 6. In this example, the bioreactor design may have been selected due to the size of the phantom shown in FIG. 6, the number of inlet(s)/outlet(s), the position of the inlet(s)/outlet(s), perfusion parameters, analyses to be performed, among others, or a combination thereof.

As shown in FIG. 8A, the bioreactor 810 may include a chamber 820 to hold the phantom and to be in fluid communication with the phantom. The chamber 820 may be bordered by an interface 834 that can be configured to be filled with bioink when the assembly is assembled. The bioreactor 810 may include an outer housing 830 and a support frame (space) 832 disposed between the interface 834 and the outer housing 830.

As shown, the bioreactor 810 may include the same number of inlet(s)/outlet(s) as the number of inlet(s)/outlet(s) of the phantom 640. For example, the outlets 842 and 844 and respective openings may correspond to the outlets 652 and 654 and respective openings of the segments 552 and 554, and the inlet 846 and respective opening may correspond to the inlet 656 as shown in FIG. 6. In some embodiments, the bioreactor may include connectors 852, 854, and 856 disposed at the openings disposed at the outlet 842, the outlet 844, and the inlet 846, respectively. One or more of the connectors 852, 854, and 856 may be barbed and configured to receive removable, corresponding barbed adapters 862, 864, and 866, respectively. In some embodiments, the connectors and/or adapters may omit or not include barbs (e.g., threads), as shown in FIG. 8B.

When attached, the adapters and/or connectors may be configured to provide strain relief to minimize damage to the bioprinted phantom. The adapters and connectors may also provide a more straightforward coupling and decoupling with perfusion systems that are readily available. The type (e.g., configuration of barbs/treads (amount, omission (see FIG. 8B), length, diameter, etc.)) of the connections/adapters may be based on the type of inlet(s)/outlet(s), size of inlet(s)/outlet(s), among others, or a combination thereof. For example, an outlet that has the largest diameter may be considered primary and the other outlets may be considered to be secondary.

Next, the method 700 may include a step 720 of modifying the dimensions of the bioreactor chamber and/or interface based on the phantom dimensions. For example, the dimensions of the chamber 820 and/or the interface 834 should be scaled so that a minimal amount of space surrounds the phantom. By way of example, the size of the interface may be preset by the user and/or the system to be a set amount of the phantom and/or fixed percentage of the phantom.

Next, the method may include a step 730 of modifying the inlet(s)/outlet(s) of the bioreactor based on the phantom model. For example, the diameter of the inlet(s)/outlet(s) may be adjusted to correspond to the size of the inlets/outlets of the phantom. By way another example, the position of the inlet(s)/outlet(s) of the bioreactor with regards to the size may be adjusted, the length of the inlet(s)/outlet(s) of the bioreactor with respect to the chamber may be adjusted, among others, or a combination thereof.

In some embodiments, the (barbed) connections and adapters may be simultaneously determined when determining bioreactor design. In some embodiments, the (barbed) connections and adapters may be determined/modified when modifying the inlet(s)/outlet(s) of the bioreactor based on the phantom.

For example, the system may use the model of the phantom to modify the bioreactor inlet(s)/outlet(s). For example, the optimal orientation of the model of the phantom with respect to the bioreactor may be determined, for example, such that the phantom and bioreactor substantially fall on the same plane (for example, as shown in FIG. 8A). After which, the position, length, among others, of the inlet(s)/outlet(s) of the bioreactor template may be modified to align to and match the location of the respective inlet(s)/outlet(s) of the phantom model. In some embodiments, the cover of the bioreactor may be modified based on the (modified) inlet(s)/outlet(s).

For example, as shown in an example 870 of FIG. 8B, the inlet 846 may be adjusted to match the internal diameter of the vessel that would be used as flow access in the phantom. The outlets 842 and 844 may be adjusted by matching them to the vessel diameters that mimic the outflow from the phantom. As shown in FIG. 8B, an additional segment 876 may be added to the inlet 846 and the outlet 844. The additional segments 876 may be configured to keep that vessel (e.g., bioprinted segment of the phantom when assembled and attached to a perfusion system) open.

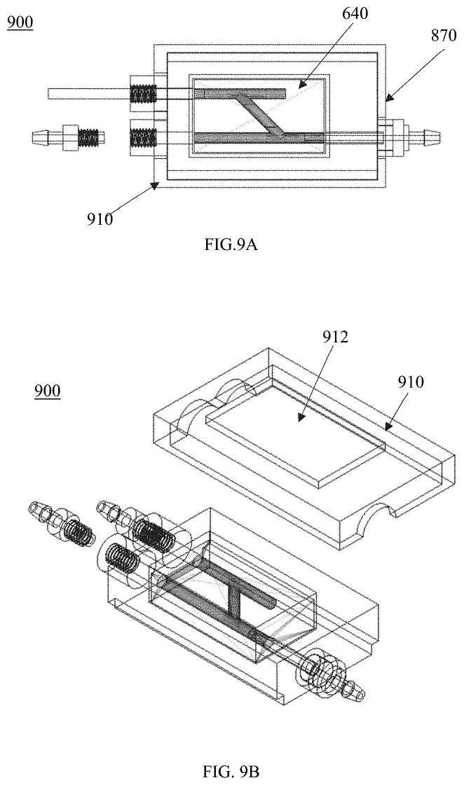

By way of example, FIGS. 9A and 9B show example of a model of the generated assembly (after step 230). FIG. 9A shows an assembly 900 that includes the phantom 640, the bioreactor 870 and a (modular) bioreactor cover 910. As shown, the modular bioreactor cover 910 may include a window 912 so that the phantom 640 of the vascular network can be visible. FIG. 9B shows an exploded view of the assembly 900 wherein the cover 910 is removed from the assembled bioreactor and phantom. As shown, the cover 910 may be designed to be tailored to the number, size and/or location of inlet(s)/outlet(s). For example, as shown, the cover 910 may include shaped openings (e.g., half-moon shaped) that correspond to the inlet 846/656, the outlet 842/652 and the outlet 844/654 and/or respective connectors 852, 854, and 856.

After the geometric model of the bioreactor is generated, the method 200 may optionally further include a step 240 of optimizing the phantom, bioreactor, and/or assembly according to some embodiments. For example, the optimization may be performed using the geometric models, using the produced models, among others, or a combination thereof.

In some embodiments, the step 240 may include producing the phantom, the bioreactor, and/or assembly using the one or more 3D printers, and test the phantom, the bioreactor and/or assembly to determine optimization. In some embodiments, the phantom and the bioreactor may be individually produced. In some embodiments, the assembly of the phantom and bioreactor may be produced together.

For example, for the bioreactor, the design and print resolution may be evaluated for optimization. By way of example, the step 240 may include producing the bioreactor using one or more 3D printers and testing the incorporated threads, viewing window, disassembly method, among others, or any combination thereof.

For example, for the phantom, to evaluate the dimensions for fit with the bioreactor, the phantom may be 3D printed in resin. In some embodiments, the vessel resolution allowing for open channels may also be validated, weak areas or regions prone to delamination due to geometry may be identified, among others, or a combination thereof.

For example, for the assembly, after producing and assembling the bioreactor and (resin) phantom, the assembly may be tested by connecting the assembly to a media source. For example, the assembly may be perfused using the media source so that the desired flow rates may be evaluated. Also, the interface between the phantom and bioreactor may be evaluated to ensure proper alignment of the phantom and bioreactor, stability of the interface at desired flow rates, and that gaps between the phantom and bioreactor housing are sufficient to place the phantom in the housing and allow for bioink backfill.

In some examples, the phantom may be bioprinted using bioink to evaluate and optimize formulation and cross-linking parameters of the bioink.

In some examples, the phantom may be bioprinted in more than one candidate orientation to select the orientation that has minimal cross-linking area. For example, the phantom may be bioprinted in more than one pre-determined orientations. For example, the orientations may be evaluated with respect to potential delamination regions, bioink issues (e.g., drying out), artifacts within the vascular network, among others, or a combination thereof.

In some examples, if the assembly is to be used for cellularized assays, the assembly may be seeded with the target cells before testing. For example, the channels of the assembly may be pre-coated with adhesion molecules (laminin, gelatin, cadherin, etc.) before seeding with target cells (endothelial cells (ECs), smooth muscle cells (SMCs), Fibroblasts, etc.) into the bioprinted phantom.

In another example, for the assembly, after producing the bioreactor and bioprinted phantom, the bioreactor and bioprinted phantom may be assembled and evaluated. For example, the bioprinted phantoms may be placed in the bioreactor enclosure and aligned so that the inlet(s)/outlet(s) openings match. After aligned, the phantom may be immobilized with temporary pins and the interface (e.g., empty space between the phantom and bioreactor) may be backfilled with bioink that is cross-linked to stabilize the bioprinted phantom for flow experiments.

After assembled, the design and/or stability of the assembly may be evaluated with respect to homeostatic flow rates. For example, the assembly may be hooked up to peristaltic pump and perfused at homeostatic flow rate. After which, the assembly may be inspected for interface leaks, phantom break up, or excessive bubble trapping is conducted during the initial validation flow assay.

If the method 200 includes step 240, in some examples, the steps 220 and 230 may be repeated if necessary to optimize the assembly.

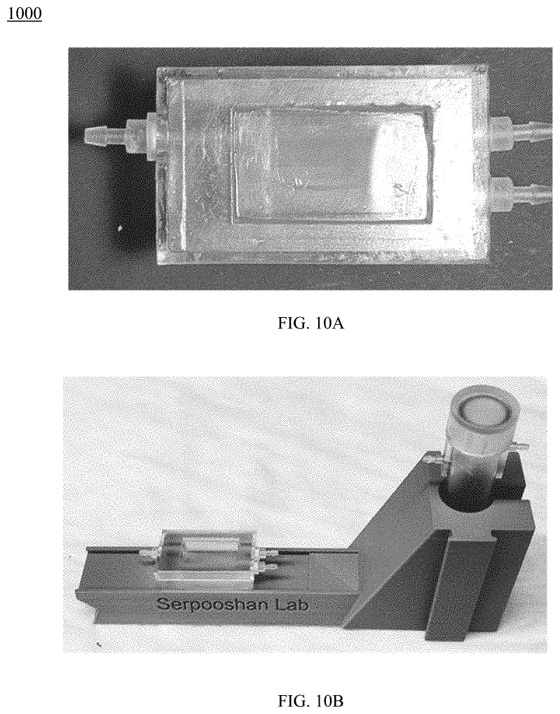

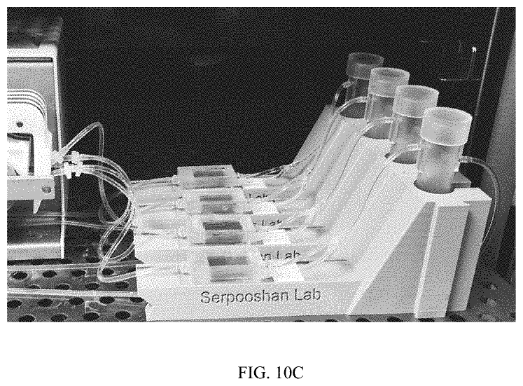

In some embodiments, the method 200 may include a step 250 of producing the 3D perfusable assembly (to be used in a system for analysis) after the step 230 and/or the step 240. FIG. 10A shows an example of the produced assembly 1000 that corresponds to the models shown in FIGS. 9A and 9B.

For example, the phantom and bioreactor may individually be produced. By way of example, the phantom may be bioprinted using the bioink parameters, the bioreactor may be 3D printed in resin, and the phantom and bioreactor may be assembled after the printing. For example, after producing the 3D printed bioreactor and bioprinted phantom, the bioprinted phantom may be placed in the bioreactor chamber and aligned so that the inlet(s)/outlet(s) openings match. After alignment, the interface between the phantom and bioreactor may be backfilled with bioink that is then cross-linked via light, chemical, time, or a combination thereof methods.

In another example, the phantom and bioreactor may be produced together using a 3D printer having bioprinting capabilities.

After the assembly is produced, the method 200 may include a step 260 of performing one or more analyses using the assembly. For example, the analyses may include but are not limited to downstream perfusion assays; in situ analyses; cellular analyses; metabolic activity analyses; bioprofiling secretion profile analyses; IHC/ICC marker staining analyses; among others; or any combination thereof. For example, the assembly 1000 may be connected to a perfusion system (e.g., tubing and peristaltic pump assembly and a media source), for example, as shown in FIG. 10B or a number of the assemblies 1000 may each be connected to a perfusion system in series, for example, as shown in FIG. 10C.

For example, for downstream perfusion assays, a number of the assemblies may be connected to a perfusion system (e.g., tubing and peristaltic pump assembly and a media source) so that the phantom may be analyzed over a period of time with respect to perfusion, for example, as shown in FIG. 10C.

For example, for in situ analyses (e.g., metabolic activity, bioprofiling, secreted proteins and molecules, etc.), a number of the assemblies may be connected to a perfusion system (e.g., tubing and peristaltic pump assembly) and a media source. At specific timepoints, during a period of time, supernatant for bioprofiling and/or secreted small molecules and proteins may be removed and analyzed. Also, metabolic activity dye may be added to the media on the day of readout and collected after appropriate incubation from the assembly while the device is under flow.

In some examples, for cellular analyses, each assembly may be seeded with the target cells before connected to the media source. After the period of time, the cellularized phantom may be removed from the assembly for analyses. For example, the cellularized phantoms may be used for imaging applications (e.g., IHC/ICC staining and imaging).

In some examples, for analysis of metabolic activity, bioprofiling secretion profile, and IHC/ICC marker staining, sectioned phantoms may be stained using commercially available antibodies according to known methods and imaged on a confocal for assay-specific marker expression and localization.

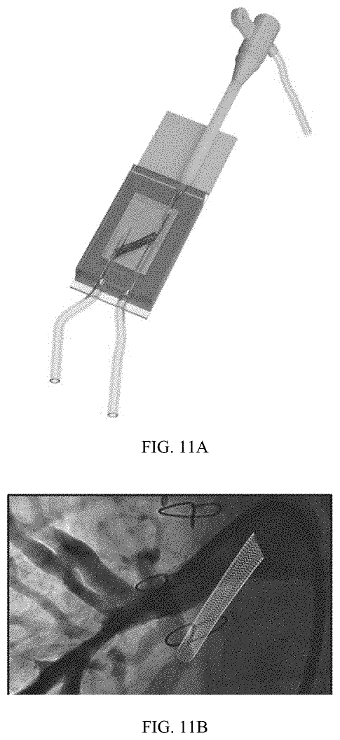

In another example, the assembly may be used to analyze an interventional procedure. For example, FIG. 11A shows an example of using a produced assembly having the model shown in FIG. 9A to test an anastomotic procedure for PA. In this example, the 3D bioprinted model may be used to recapitulate the vascular atresia based on the anatomical data obtained from Tetralogy of Fallot (TOF) with major aortopulmonary collateral arteries (MAPCAs) patient (shown in FIG. 3) as shown in FIG. 11B.

In some embodiments, the plurality of assemblies connected in parallel may be the same or different. For example, the assemblies may be different so as to analyze different geometrical, structural, biomechanical, and/or flow parameters in the vascular phantom (e.g., diameter, location, and angle of conduit, blood velocity, and elasticity/stiffness). By way of example, the conduit segment in the phantom assemblies may differ with respect to angle, location and/or diameter. By evaluating a variety of connection designs and/or angles, phantoms that cause pathologic turbulent flows that could eventually close the newly cleared conduit could be identified so that the treatment based on those phantoms could be avoided.

One or more of the devices and/or systems of the system 100 may be and/or include a computer system and/or device. FIG. 12 is a block diagram showing an example of a computer system 1200. The modules of the computer system 1200 may be included in at least some of the systems and/or modules, as well as other devices and/or systems of the system 100.

The system for carrying out the embodiments of the methods disclosed herein is not limited to the systems shown in FIGS. 1 and 12. Other systems may also be used. It is also to be understood that the system 1200 may omit any of the modules illustrated and/or may include additional modules not shown.

The system 1200 shown in FIG. 12 may include any number of modules that communicate with each other through electrical or data connections (not shown). In some embodiments, the modules may be connected via any network (e.g., wired network, wireless network, or any combination thereof).

The system 1200 may be a computing system, such as a workstation, computer, or the like. The system 1200 may include one or more processors 1212. The processor(s) 1212 may include one or more processing units, which may be any known processor or a microprocessor. For example, the processor(s) may include any known central processing unit (CPU), graphical processing unit (GPU) (e.g., capable of efficient arithmetic on large matrices encountered in deep learning models/classifiers), among others, or any combination thereof. The processor(s) 1212 may be coupled directly or indirectly to one or more computer-readable storage media (e.g., memory) 1214. The memory 1214 may include random access memory (RAM), read only memory (ROM), disk drive, tape drive, etc., or any combinations thereof. The memory 1214 may be configured to store programs and data, including data structures. In some embodiments, the memory 1214 may also include a frame buffer for storing data arrays.

In some embodiments, another computer system may assume the data analysis, image processing, or other functions of the processor(s) 1212. In response to commands received from an input device, the programs or data stored in the memory 1214 may be archived in long term storage or may be further processed by the processor and presented on a display.

In some embodiments, the system 1200 may include a communication interface 1216 configured to conduct receiving and transmitting of data between other modules on the system and/or network. The communication interface 1216 may be a wired and/or wireless interface, a switched circuit wireless interface, a network of data processing devices, such as LAN, WAN, the internet, or any combination thereof. The communication interface may be configured to execute various communication protocols, such as Bluetooth, wireless, and Ethernet, in order to establish and maintain communication with at least another module on the network.

In some embodiments, the system 1210 may include an input/output interface 1218 configured for receiving information from one or more input devices 1220 (e.g., a keyboard, a mouse, and the like) and/or conveying information to one or more output devices 1220 (e.g., a printer, a CD writer, a DVD writer, portable flash memory, etc.). In some embodiments, the one or more input devices 1220 may be configured to control, for example, the generation of the management plan and/or prompt, the display of the management plan and/or prompt on a display, the printing of the management plan and/or prompt by a printer interface, the transmission of a management plan and/or prompt, among other things.

In some embodiments, the disclosed methods (e.g., FIGS. 2, 4 and 7) may be implemented using software applications that are stored in a memory and executed by the one or more processors (e.g., CPU and/or GPU) provided on the system 100. In some embodiments, the disclosed methods may be implemented using software applications that are stored in memories and executed by the one or more processors distributed across the system.

As such, any of the systems and/or modules of the system 100 may be a general purpose computer system, such as system 1200, that becomes a specific purpose computer system when executing the routines and methods of the disclosure. The systems and/or modules of the system 100 may also include an operating system and micro instruction code. The various processes and functions described herein may either be part of the micro instruction code or part of the application program or routine (or any combination thereof) that is executed via the operating system.

If written in a programming language conforming to a recognized standard, sequences of instructions designed to implement the methods may be compiled for execution on a variety of hardware systems and for interface to a variety of operating systems. In addition, embodiments are not described with reference to any particular programming language. It will be appreciated that a variety of programming languages may be used to implement embodiments of the disclosure. An example of hardware for performing the described functions is shown in FIGS. 1 and 12. It is to be further understood that, because some of the constituent system components and method steps depicted in the accompanying figures can be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed:

1. A method for generating a 3D perfusable assembly of a vascular network, comprising:
acquiring one or more images of an anatomy of interest, the anatomy of interest including a target area;
generating a geometric model of a phantom of a vascular network using the one or more images;
the phantom including one or more vascular segments, one or more inlets, and one or more outlets, each inlet and each outlet communicating with at least one vascular segment;
generating a geometric model of a bioreactor based on the geometric model of the phantom using one or more of assembly parameters, phantom parameters, or any combination thereof; and
the bioreactor model including one or more inlets, one or more outlets, a chamber in which the phantom is disposed, an outer housing, and an interface bordering the chamber.

2. The method according to claim 1, further comprising:
producing the phantom and/or the bioreactor using a 3D dimensional printer.

3. The method according to claim 2, wherein the phantom is bioprinted using bioink and the bioreactor is printed using resin.

4. The method according to claim 3, wherein the interface is configured to be filled with bioink during assembly of the phantom and the bioreactor.

5. The method according to claim 1, wherein the generating the geometric model of a bioreactor includes:
selecting a bioreactor template from a plurality of stored bioreactor templates using one or more assembly parameters, generated phantom parameters, among others, or a combination thereof; and
modifying the bioreactor template to correspond to at least the geometric model of the phantom.

6. The method according to claim 5, wherein:
the modifying includes adjusting the interface based on dimensions of the phantom, one or more bioreactor settings, among others, or a combination thereof; and
the interface being configured to be filled with bioink when the bioreactor and the phantom are assembled.

7. The method according to claim 1, wherein the vascular network includes a conduit segment in fluid communication with the one more vascular segments, the conduit segment representing a treatment site.

8. The method according to claim 7, wherein the vascular network includes one or more vascular segments representing pulmonary artery stenosis.

9. The method according to claim 7, wherein the conduit segment is connected to the one or more of vascular segments at a location of a proposed treatment.

10. The method according to claim 1, wherein the generating a geometric model of the phantom includes:
identifying the one or more of the vascular segments of the target area based on active flow regions within the target area using at least clinical data; and
removing one or more other vascular segments that are outside of the active flow regions.

11. A system for generating a 3D perfusable assembly of a vascular network, comprising:
one or more processors; and
one or more hardware storage devices having stored thereon computer-executable instructions which are executable by the one or more processors to cause the computing system to perform at least the following:
acquiring one or more images of an anatomy of interest, the anatomy of interest including a target area;
generating a geometric model of a phantom of a vascular network using the one or more images;
the phantom including one or more vascular segments, one or more inlets, and one or more outlets, each inlet and each outlet communicating with at least one vascular segment;
generating a geometric model of a bioreactor based on the geometric model of the phantom using one or more of assembly parameters, phantom parameters, or any combination thereof; and
the bioreactor model including one or more inlets, one or more outlets, a chamber in which the phantom is disposed, an outer housing, and an interface bordering the chamber.

12. The system according to claim 11, wherein the one or more processors are further configured to cause the computing system to perform at least the following:
producing the phantom and/or the bioreactor using a 3D dimensional printer.

13. The system according to claim 12, wherein the phantom is bioprinted using bioink and the bioreactor is printed using resin.

14. The system according to claim 13, wherein the interface is configured to be filled with bioink during assembly of the phantom and the bioreactor.

15. The system according to claim 11, wherein the generating the geometric model of a bioreactor includes:
selecting a bioreactor template from a plurality of stored bioreactor templates using one or more assembly parameters, generated phantom parameters, among others, or a combination thereof; and
modifying the bioreactor template to correspond to at least the geometric model of the phantom.

16. The system according to claim 15, wherein:
the modifying includes adjusting the interface based on dimensions of the phantom, one or more bioreactor settings, among others, or a combination thereof; and
the interface being configured to be filled with bioink when the bioreactor and the phantom are assembled.

17. The system according to claim 11, wherein the vascular network includes a conduit segment in fluid communication with the one more vascular segments, the conduit segment representing a treatment site.

18. The system according to claim 17, wherein the vascular network includes one or more vascular segments representing pulmonary artery stenosis.

19. The system according to claim 17, wherein the conduit segment is connected to the one or more of vascular segments at a location of a proposed treatment.

20. The system according to claim 11, wherein the generating a geometric model of the phantom includes:
identifying the one or more of the vascular segments of the target area based on active flow regions within the target area using at least clinical data; and
removing one or more other vascular segments that are outside of the active flow regions.
